(19) United States
(12) Patent Application Publication
(10) Pub. No.: US 2010/0266650 A1
(43) Pub. Date: Oct. 21, 2010

(54) METHOD OF COSMETIC CARE STIMULATING MITOCHONDRIAL ACONITASE

(75) Inventors: Carine NIZARD, Ivry Sur Seine (FR); Marielle Moreau, Marcq (FR); Jean-Christophe Archambault, Meung Sur Loire (FR)

Correspondence Address: MERCHANT & GOULD PC, P.O. BOX 2903, MINNEAPOLIS, MN, 55402-0903, US

(73) Assignee: LVMH RECHERCHE, Saint Jean de Braye (FR)

(21) Appl. No.: 12/751,710

(22) Filed: Mar. 31, 2010

(30) Foreign Application Priority Data
Mar. 31, 2009 (FR) 0952072

(51) Int. Cl. A61K 8/02; A61K 8/97; A61Q 19/00

(52) U.S. Cl. 424/401; 424/736

(57) ABSTRACT

The invention relates to the use of a plant extract as a cosmetic agent.

This extract is obtained from a plant belonging to the genus Citrus, or from hybrids derived from the crossing of plant species, at least one of which belongs to the genus Citrus, as a cosmetic active agent.

The invention makes it possible to prevent or retard the appearance of the signs of ageing of the skin or to attenuate the effects thereof and/or to improve or restore the radiance of the skin complexion.

This application claims the benefit of Serial No. 0952072, filed Mar. 31, 2009 in France and which application(s) are incorporated herein by reference. A claim of priority to all, to the extent appropriate is made.

The present invention relates to a novel use of a plant extract obtained from at least one plant belonging to the genus Citrus, or a hybrid obtained from the crossing of plant species, at least one of which belongs to the genus Citrus, as a cosmetic agent more particularly for producing an anti-ageing effect on the skin.

The invention is more particularly directed towards an extract of a plant of the genus×Citrofortunella, for example an extract of calamondin (×Citrofortunella microcarpa), as a cosmetic active agent, and also to anti-ageing cosmetic compositions comprising the said extract and to cosmetic methods using the said compositions for stimulating the activity of mitochondrial aconitase and for obtaining an anti-ageing effect on the skin.

### PRIOR ART

Ageing is a multi-factor phenomenon. Several theories exist regarding ageing, among which is the free radical theory based on the chemical nature and ubiquitous presence of these radicals (Harman D., J. Gerontol., 1956; 11, 298-300).

These free radicals, also known as reactive oxygen species (ROS), may be of exogenous origin or produced via various cellular processes, especially during mitochondrial respiration (Cadenas E. et al., Free Radic. Biol. Med., 2000; 29, 222-230).

During respiration, a small but significant amount of the total oxygen consumed by the respiratory chain is converted into superoxide radical O₂²⁻, which may lead to the formation of other even more reactive oxygen species, for instance hydrogen peroxide H₂O₂, and hydroxyl and peroxynitrite radicals (cf. Cadenas et al., cited above).

Under conditions of oxidative stress, the formation of these reactive species results in oxidative damage to proteins, DNA and lipids, and also to changes in the expression of the mitochondrial proteins and contributes towards the process of ageing of the skin (Bulteau et al., Exp. Gerontol., 2006, 41; 653-657).

During ageing, reduced efficacy of the system for maintenance of cellular macromolecules and a constant increase in the production of ROS in the mitochondria are observed (Humphries et al., Free Radic. Res., 2006; 40, 1239-1243).

Now, the mitochondrion plays an important role in many cell functions, including the production of the proton gradient established by the respiratory chain and the production of ATP via the Krebs cycle (Liu et al., J. Neurochem., 2002; 80, 780-787).

Accumulation of oxidative damage and structural anomalies brings about in the mitochondrion the gradual loss of its capacity to produce the ATP required for the functioning and integrity of the cell (Frenzel et al., 1984).

Aconitase is an essential mitochondrial enzyme of the Krebs cycle, which converts citrate into isocitrate. It also plays a role in the preservation of mitochondrial DNA. By means of aconitase, the stability and hereditary transmission of this DNA are thus closely linked to the metabolic state of the cell (Chen et al., Proc. Natl. Acad. Sci. USA, 2007; 104, 13738-13743).

The activity of aconitase depends on the integrity of its iron-sulfur centre [4Fe-4S]²⁺ (Beiner, et al., Faseb J, 1993; 7, 1442-1449). Attack of its iron-sulfur centre [4Fe-4S]²⁺ by oxidizing agents brings about the formation of an iron-sulfur centre [3Fe-4S]⁺ which inactivates the aconitase.

The loss of activity of mitochondrial aconitase is an intracellular indicator of oxidative damage and cell ageing. Many degenerative disorders are also associated with the increase in the levels of pro-oxidative agents and the drop in activity of aconitase in the mitochondrion (Bulteau et al., Biochemistry, 2003, 42, 14846-14855).

Inactivation of aconitase especially brings about a change in the NADH/NAD⁺ ratios. Specifically, the production of NADH by α-ketoglutarate dehydrogenase and isocitrate dehydrogenase will be lowered in the Krebs cycle due to the fall in activity of aconitase (cf. Humphries et al., cited above; Nulton-Persson et al., J. Biol. Chem., 2001, 276, 23357-23361). Under these conditions of NAD⁺ accumulation, an increase in ROS is observed on account of the autoxidation of the reduced metabolites. Such an inactivation may also initiate a cascade of oxidative reactions that contributes towards the accumulation of damaged proteins (cf. Humphries et al., 2006, cited above).

It is thus essential, during ageing, to maintain a sufficient degree of activity of mitochondrial aconitase in skin cells, in order to prevent the accumulation of such cell damage and to promote the processes of repair and reactivation of the aconitase damaged by these ROS.

### SUMMARY OF THE INVENTION

The inventors of the present invention have demonstrated that the activity of mitochondrial aconitase decreases by about 85% in human dermal fibroblasts in culture obtained from donors 70 years old, in comparison with those obtained from donors 20 years old, without any change in expression of the protein with age. Starting from this finding, they demonstrated that it is possible to totally protect mitochondrial aconitase and to re-establish its activity in these aged fibroblasts in culture, by treating the said fibroblasts with a plant extract obtained from at least one plant belonging to the genus Citrus or a hybrid obtained from the crossing of plant species, at least one of which belongs to the genus Citrus, and more particularly an extract of calamondin, whereas this same treatment applied to the fibroblasts of a young donor does not modify the activity of the mitochondrial aconitase.

Such an effect on stimulating the activity of mitochondrial aconitase enables this enzyme to conserve its central role in the Krebs cycle and in the preservation of the mitochondrial DNA, and also to preserve the functioning, especially the metabolic functioning, of skin cells.

This results from re-establishment of the activity of the mitochondrial aconitase of the skin cells of elderly individuals, an effect of slowing down the ageing of skin cells being reflected by an anti-ageing cosmetic effect.

Moreover, the inventors have also demonstrated that the abovementioned extract allows a significant reduction in the levels of intracellular oxidized proteins, which increases with the age of the donors, such that this increase is totally reversed by treating these cells with a plant extract as defined previously.

This antioxidant activity of the extract of the invention, for which the link with the activity of mitochondrial aconitase is not established, gives the extract of the invention particularly advantageous additional properties in the cosmetics field as a cosmetic active agent for improving or restoring the radiance of the skin complexion.

Specifically, oxidized proteins have different optical properties from normal proteins. The technique of circular dichroism has shown that the light transmitted by an oxidized protein is different from that transmitted by a normal protein (Friguet et al., FEBS Lett, 1997, 405(1): 21-5). As a result, the accumulation of oxidized proteins has an impact on the skin, and in particular on the skin complexion, by causing deregulation of the cutaneous cell cycle, resulting in an impairment in its natural genetically determined coloration, and on the visual perception of the skin by an observer.

Studies (not published to date) have been conducted by the Applicant to investigate, using a glossmeter (Samba machine supplied by the company Bossa Nova Technologie) on a model of reconstructed skin, the reflection of light on the skin and the influence of oxidation on the light reflection. It has thus been shown that untreated skin has a higher total light reflection than skin that has been oxidized (measurement by glossmeter).

The genus×Citrofortunella includes plant species derived from the crossing of plants of the genus Citrus with those of the genus Fortunella.

Among the plant species belonging to the genus Citrofortunella, mention may be made in a non-limiting manner and by way of example of ×Citrofortunella fiondana, ×Citrofortunella microcarpa or ×Citrofortunella mitis.

Among these plant species, calamondin (×Citrofortunella microcarpa), also referred to without preference as kalamansi or Citrus madurensis, is advantageously chosen.

Japanese patent application JP 2003-199 527 discloses a food that inhibits the increase of glycaemia or blood pressure, the said food being characterized in that it contains an extract of the whole fruit of a hybrid of a citrus and of a kumquat of the genus Fortunella.

Also, Japanese patent application JP 2005-029 491 discloses a method for extracting limonene via fractional distillation of the pericarp of Citrofortunella mitis.

To date, no disclosure has been made of the noteworthy activity of a plant extract obtained from at least one plant belonging to the genus Citrus or a hybrid derived from the crossing of plant species, at least one of which belongs to the genus Citrus, for example to the genus×Citrofortunella, and in particular an extract of calamondin (×Citrofortunella microcarpa) with respect to the oxidized proteins of skin cells and mitochondrial aconitase.

Calamondin extract thus constitutes a novel active agent in the field of cosmetics, and especially as a cosmetic active agent in a cosmetic composition for preventing, slowing down or attenuating the effects of ageing of the skin, especially following the application of an oxidative stress to the skin.

### AIMS OF THE INVENTION

A main aim of the invention is especially to provide a novel use of a plant extract obtained from at least one plant belonging to the genus Citrus or a hybrid derived from the crossing of plant species, at least one of which belongs to the genus Citrus, as a cosmetically acceptable active agent, more particularly for producing an anti-ageing effect on the skin and/or for caring for damaged skin, especially skin damaged by ultraviolet radiation, and/or for improving or restoring the radiance of the skin complexion.

The invention is more particularly directed towards a novel use of an extract of a plant belonging to the genus×Citrofortunella, and especially an extract of calamondin (×Citrofortunella microcarpa), as a novel cosmetic active agent, to its use as an active agent in a cosmetic composition, and to a cosmetic care method using the said composition.

A main aim of the present invention is also a novel use of an extract of calamondin (×Citrofortunella microcarpa) as an active agent in a cosmetic composition comprising at least one cosmetically acceptable excipient.

This novel use is directed in particular towards preventing or retarding the appearance of the signs of ageing of the skin or for treating them, especially associated with damage caused by an excess of ROS, or for producing an anti-ageing effect on the skin.

This novel use is also directed towards improving or restoring the radiance of the skin complexion.

A main aim of the invention is also a cosmetic method using the said extract in a cosmetic composition for the purpose of preventing or slowing down the effects of ageing of the skin and/or for improving or restoring the radiance of the skin complexion, by applying the said composition comprising the extract according to the invention to at least a concerned part of the body or the face.

A main aim of the invention is also to propose the use of an extract of calamondin, as an active agent in an anti-ageing cosmetic composition and/or as an active agent in a composition for improving or restoring the radiance of the skin complexion.

An aim of the invention is also the use of the said extract as an active agent in cosmetic compositions, and cosmetic care methods using the said compositions for preventing or retarding the appearance of the signs of ageing of the skin or for attenuating the effects thereof and/or for improving or restoring the radiance of the skin complexion.

A main aim of the invention is also to provide a cosmetic care method, using the said extract, especially to perform the cosmetic care indicated above.

### DESCRIPTION OF THE INVENTION

A first subject of the present invention is thus directed towards a novel use of a plant extract obtained from at least one plant belonging to the genus Citrus or a hybrid derived from the crossing of plant species, at least one of which belongs to the genus Citrus, as a cosmetically acceptable active agent, more particularly for producing an anti-ageing effect on the skin and/or for caring for damaged skin, especially skin damaged by ultraviolet radiation, and/or for improving or restoring the radiance of the skin complexion.

Among the hybrids that are particularly preferred are those belonging to the genus×Citrofortunella, resulting from the crossing of plant species of the genus Citrus with plant species belonging to the genus Fortunella, and more particularly calamondin (×Citrofortunella microcarpa).

Another subject of the present invention is also directed towards a novel use of an extract of calamondin as a cosmetic active agent, more particularly for producing an anti-ageing effect on the skin and/or for improving or restoring the radiance of the skin complexion.

The plant material used for the preparation of the extract may be the whole plant or a part of the plant such as the root, the rhizome or an aerial part, especially the stem, the leaves, the flowers, the seeds, the fruit or the floral buds.

It may advantageously be formed from the whole fruit or a part of the fruit of one of the species mentioned above.

A preferred extract is obtained from calamondin fruit.

Before the extraction step per se, the plant material may have been dried and/or ground, or alternatively may be in the freshly harvested state.

The extract may be prepared via various extraction processes known to those skilled in the art.

The extraction may be performed without solvent, for example by pressing, especially of a whole fruit or of part of a fruit.

However, the extraction is advantageously performed by placing the selected plant material in contact with a polar solvent or a mixture of polar solvents, especially by soaking, maceration or decoction of the said plant material in the appropriate solvent or solvent mixture.

According to one particularly advantageous variant, the extract is obtained from a fruit juice of these plants, especially obtained by pressing, by placing in contact with a polar solvent or a mixture of polar solvents, preferably an aqueous medium.

The polar solvent or mixture of polar solvents used for the extraction is advantageously chosen from water, a C1-C4 alcohol, in particular chosen from ethanol and butanol, a glycol preferentially chosen from glycerol, butylene glycol and propylene glycol, and polyglycerol-3, and mixtures thereof.

The preferred mixtures are mixtures of at least one alcohol and water or of at least one glycol and water, comprising at least 10 v/v % of alcohol or glycol, the remainder being formed from water.

According to another particular variant of the invention, the solvent mixture comprises a mixture of water and ethanol in a 50/50 v/v ratio, or a mixture of water and butylene glycol in a 50/50 v/v ratio.

According to one preferred embodiment, the extraction is advantageously performed in aqueous or water-glycol medium.

The extraction may also optionally comprise an additional step that consists of a treatment of the plant material or plant extract, aimed at partially or totally decolorizing it or purifying it, for example via a treatment of the plant material or the extract with a solution of an apolar solvent or solvent mixture or via a treatment that consists in placing the extract in contact with active charcoal particles, or alternatively via a treatment with supercritical CO₂.

The extraction may be completed by a step of partial or total removal of the extraction solvents. In the first case, the extract is generally concentrated until an aqueous concentrate freed of significant amounts of organic solvent is obtained, and in the second case, a dry residue is obtained. Alternatively, the product from the extraction step may be freeze-dried or atomized in the form of a powder.

The powder may be used in the form as obtained, in a cosmetic composition according to the invention, or may be dispersed or dissolved in a polar solvent or a mixture of polar solvents, or alternatively may be adsorbed onto a solid support.

According to one embodiment variant of the present invention, the abovementioned active agent is delivered topically incorporated into a cosmetic composition, the said active agent being present in an amount that is effective for preventing or retarding the signs of ageing of the skin, or for attenuating the effects thereof; and/or for caring for damaged skin, especially skin damaged by ultraviolet radiation, and/or for improving or restoring the radiance of the skin complexion.

The said active agent may also be mixed with at least one cosmetically acceptable excipient, and may be used by applying this composition to bodily or facial skin.

Thus, the invention is also directed towards a cosmetic composition comprising at least one cosmetically acceptable active agent and at least one cosmetically acceptable excipient, characterized in that the said active agent is a plant extract obtained from at least one plant belonging to the genus Citrus or a hybrid derived from the crossing of species, at least one of which belongs to the genus Citrus, especially a plant belonging to the genus×Citrofortunella.

The cosmetic composition according to the invention comprises an amount of the extract of the invention that is effective for obtaining the desired effect.

For any aspect of the invention, the term “effective amount” means an amount that is at least equal to the amount needed to prevent or retard the appearance of the signs of ageing of the skin or to attenuate the effects thereof; and/or to care for damaged skin, especially skin damaged by ultraviolet radiation; and/or to improve or restore the radiance of the skin complexion.

The cosmetic composition according to the invention advantageously comprises from 0.001% to 5% by weight and preferably between 0.01% and 3% by weight of the composition, as active agent.

The composition may also advantageously comprise other active agents that have cosmetic effects similar and/or complementary to those of the invention, especially at least one other active agent that participates in the maintenance and/or integrity of the structure of the skin, and at least one cosmetically acceptable excipient that may be chosen especially from pigments, dyes, polymers, surfactants, rheological agents, fragrances, electrolytes, pH modifiers, antioxidants and preserving agents, and mixtures thereof.

The cosmetic composition according to the invention may be formulated, for example, in the form of a serum, a lotion, an emulsion, for example a cream, or alternatively a hydrogel, preferably a mask, or may be in the form of a stick or a patch.

Finally, the present invention relates to a use of the active agents as defined above as cosmetic active agents for preventing or retarding the appearance of the signs of ageing of the skin or for treating them, the said cosmetic agent stimulating the activity of the mitochondrial aconitase of skin cells.

The invention also relates to a use of the cosmetic active agent of the invention for the manufacture of a cosmetic composition for preventing or retarding the appearance of the signs of ageing of the skin or for attenuating the effects thereof and/or for improving or restoring the radiance of the skin complexion.

Finally, the invention is directed towards a cosmetic care method for preventing or retarding the appearance of the signs of ageing of the skin or for attenuating the effects thereof and/or for caring for damaged skin, especially skin damaged by ultraviolet radiation and/or for improving or restoring the radiance of the skin complexion, characterized in that it comprises the delivery to at least one concerned area of bodily or facial skin of an effective amount of at least one cosmetically acceptable active agent, the said active agent being a plant extract obtained from a plant belonging to the genus Citrus, or a hybrid derived from the crossing of plant species, at least one of which belongs to the genus Citrus, especially a plant belonging to the genus×Citrofortunella, and in particular a plant of the plant species×Citrofortunella microcarpa.

The said method is preferably characterized in that the active agent, incorporated into a cosmetic composition also comprising at least one cosmetically acceptable excipient, is topically delivered by applying the said composition to at least one concerned area of bodily or facial skin.

The care method according to the invention is also characterized in that the concentration of active agent is between 0.001% and 5% by weight of the cosmetic composition.

Other aims, characteristics and advantages of the invention will emerge clearly from the explanatory description that follows, which is given with reference to several examples of implementation of the invention that are given purely for illustrative purposes and shall not in any way constitute a limitation of the scope of the invention.

In the description, and in particular in the examples that follow, all the percentages are given on a weight basis, the temperature is in degrees Celsius, and the pressure is atmospheric pressure, unless otherwise indicated.

### DESCRIPTION OF THE FIGURES

FIG. 1 relates to the modulation of mitochondrial aconitase activity with age, measured after culturing and treating human fibroblasts obtained from donors 20 and 70 years old, according to Example 2: (A) measurement of the mitochondrial aconitase activity (cf. Ex. 2, paragraph 3); (B) mitochondrial aconitase assay by Western blotting (WB) (cf. Ex. 2, paragraph 4).

FIG. 2 relates to modifications of the active site of mitochondrial aconitase with age, measured after culturing and treating human fibroblasts obtained from donors 20 and 70 years old, according to Example 2; separation and measurement of the forms of mitochondrial aconitase by immunoelectrofocusing (IEF) and then Western blotting (Ex. 2, paragraph 5).

FIG. 3 relates to the measurement of the mitochondrial aconitase activity, measured after culturing human fibroblasts obtained from donors 20 and 70 years old, cultured according to Example 2, and then treated with a 2.5% extract of calamondin for 48 hours (cf. Ex. 2, paragraph 3).

FIG. 4 relates to the detection of oxidized proteins via oxy blotting on isolated mitochondria (Example 3) using human fibroblasts obtained from 20- and 70-year-old donors, placed in culture and then treated with a 2.5% calamondin extract, cosmetic active agents for 48 hours (cf. Ex. 2).

### EXAMPLES

### Example 1

### Preparation of a Calamondin Extract Used for Performing the Tests

The extract of calamondin (×Citrofortunella microcarpa) is prepared by pressing whole fruit of the plant.

After filtering and then centrifuging, the fruit pulp is extracted with a mixture of water and polyglycerol.

The extract obtained contains 35-45% by weight of solids. This extract is used in the form as obtained, to perform the tests of Examples 2 and 3, and to prepare cosmetic compositions, especially that of Example 4.

### Example 2

### Measurement of Mitochondrial Aconitase Activity in Human Fibroblasts

Materials and Methods

1. Culturing of the Human Fibroblasts and Treatment

Media and Reagents

Fibroblast Culture Medium

DMEM 1/ml of glucose (Gibco)

+10% SVF

+1% sodium pyruvate 100 mM (Gibco)

Stock Solutions of Active Agent

The stock solution is prepared by diluting the extract solution in

DMEM medium (weight %).
calamondin extract at 6% in the medium, i.e. about 2.4% by weight of solids.

a—Cell Culturing and Treatment

Human fibroblasts in primary culture obtained from plastic surgery of a donor 20 years old and of a donor 70 years old, at the 12th passage.

Subculturing on D0

Fibroblasts 15.10⁵ cells/dish of 75 cm² in triplicate in DMEM medium (10 ml/dish)

Treatment on D5

The stock solution is diluted in the DMEM medium to produce the concentrations mentioned below:
calamondin extract at 2.5% by weight in the medium, i.e. about 1% by weight of solids.

Recovery of the Cells on D7

Preparation of the Mitochondria

2 rinses with PBS

on a bed of ice; recovery in 2 ml of homogenization buffer.

b—Isolation of the Mitochondria

32 T75 dishes are used for each donor. The cells at confluence are washed twice with pH 7.2 PBS buffer (sodium phosphate buffer pH 7.2−0.13 M of NaCl, 3 mM of KCl, 8 mM of Na₂HPO₄ and 1.4 M of KH₂PO₄) and are detached by scraping, and then centrifuged at 1500×g at 4° C. for 5 minutes. The cell pellet is washed with the PBS buffer, recentrifuged and then placed in ice. The pellet is taken up in cold homogenization buffer (0.3 M mannitol, 0.1% BSA, 0.2 mM EDTA, 10 mM HEPES, adjusted to pH 7.4 with KOH, 5 times the pellet volume), homogenized on ice with a 2 ml glass homogenizer. The cell suspension is centrifuged at 1000×g at 4° C. for 10 minutes. The supernatant is recentrifuged at 10 000×g at 4° C. for 15 minutes. The supernatant contains the cytosolic fraction, and the pellet represents the mitochondrial fraction. The mitochondrial fraction is washed twice with the cold homogenization buffer. The protein concentration is measured according to the Bradford method.

2. Protein Assay (Bradford Method)

a—Preparation of the Calibration Range

BSA stock solution: 50 μg/ml (BIORAD; standard protein)

| Protein | | |
|---|---|---|
| (μg/tube) | BSA (μl) | H₂O (μl) |
| 0 | 0 | 800 |
| 1 | 20 | 780 |
| 2 | 40 | 760 |
| 3 | 60 | 740 |
| 4 | 80 | 720 |
| 5 | 100 | 700 |
| 6 | 120 | 680 |
| 8 | 160 | 640 |

200 μl of Coomassie Blue G250 are added to each tube. The blue is prepared extemporaneously by five-fold dilution of the stock solution.

b—Preparation of the Samples
if the protein concentration >3 mg/ml, the cell extracts are diluted 100-fold, and 100 μl of the dilution are then taken

+700 μl of milliQ water

+200 μl of blue
if the protein concentration is <3 mg/ml, 10 μl of cell extract are taken.

+790 μl of milliQ water

+200 μl of blue

c—Sample Assay

The samples are homogenized by vortexing and, after leaving to stand for 5 minutes, the optical density is then read on a spectrophotometer at a wavelength of 595 nm.

3. Assay of the Mitochondrial Aconitase Activity

a—Reagents
25 mM pH 7.5 TRIS buffer (Sigma)
sodium citrate (Sigma)
isocitrate dehydrogenase (Sigma)
MnCl₂ (Sigma)

b—Principle of the Mitochondrial Aconitase Activity Assay

The mitochondrial aconitase activity is quantified by measuring the absorbance at 340 nm in a reaction medium containing 0.2 mM NADP⁺, 5 mM of sodium citrate and one unit/ml of isocitrate dehydrogenase in 25 mM Tris-HCl plus 0.6 mM MnCl₂ and 0.05% Triton X-100.

For the assay, 50 μg of mitochondrial protein are added to 1.0 ml of reaction medium at 25° C. The measurements at 340 nm are recorded in 1 cm cells at 5-minute intervals and the mitochondrial aconitase activity is calculated according to the linear increase in absorbance at 340 nm over about 5 minutes. The activity is obtained by using a molar extinction coefficient for NADPH of 6.22×10³ M⁻¹ cm⁻¹ and by assuming the conversion by the isocitrate dehydrogenase of one molecule of citrate into one molecule of NADPH.

4. Mitochondrial Aconitase Assay by Western Blotting (WB)

a—Assay Principle

The protein electrophoresis is performed in a polyacrylamide minigel of 1 mm to 1.5 mm thickness, under denaturing and reductive conditions, in discontinuous buffer according to the Laemmli method (Nature, 1970; 227, 680). Gels containing 12% T and 2.7% C allow the low molecular weight proteins (20 to 120 kDa) to be separated. Gels containing 8% T and 2.7% C allow the high molecular weight proteins (35 to 250 kDa) to be separated.

The solutions required for producing the gels are presented in Appendix A.

Separating Gel

The gel is poured at least two hours before migration.

Pouring of the gel is performed using a pipette up to about 0.5 mm from the bottom of the comb provided for the concentrating gel. Absolute ethanol is added to the surface to obtain a uniform base line (±1 ml/gel).

Concentrating Gel

The ethanol is removed. 2.5 ml of gel are poured out using a polyethylene Pasteur transfer pipette (Biorad) and the combs are then inserted. The gel is polymerized after one hour.

b—Preparation of the Samples

The mitochondrial proteins are subjected to electophoresis on polyacrylamide gel containing 12% T under reductive Laemmli conditions. The samples (25-40 μg of protein) are reduced for 5 minutes at 100° C. in the deposition buffer. After depositing the samples and the markers, migration is performed at 200V for 1 hour in a 50 mM Tris-HCl, 100 mM glycine, 2 mM EDTA pH 8.4 buffer containing 0.1% SDS.

c—Electrophoresis

Deposition

The samples are heated at 95° C. for 5 minutes.

The volume to be deposited depends on the desired amount of protein (maximum volume=25 μl for a 1 mm gel and 40 μl for a 1.5 mm gel). The reference amount is 10 μg of protein, corresponding to 10 μl; it is then adapted according to the expression of the target protein.

The combs are removed. 200 ml of 1× migration buffer are poured onto the gels, into the central compartment between the two gels, and then into the tank up to the quarter level.

The samples are deposited using a tapered tip fitted onto the micropipette and 10 μl of prestained low molecular weight controls (Biorad, Prestained SDS-PAGE standards Low Range) or high molecular weight controls (Amersham, Full Range Rainbow).

Migration

The electrophoresis is performed at room temperature, at 200V. This electrophoresis is stopped when the migration front has left the gel (about 40 minutes of migration).

Semi-Dry Transfer of the Proteins onto Membrane

Two thick sheets of filter paper (Biorad) and cellulose membranes (Biorad) are soaked in Towbin transfer buffer (PNAS, 1979, 76 (9) 4350-4) (see Appendix B).

In the semi-dry transfer apparatus (Biorad), a sheet of wet thick filter paper is placed on the anode.

Once the migration is complete, the concentrating gel is removed and the separating gel is fitted on the cellulose membrane. The membrane comprising the gel is deposited on the sheet of filter paper. The second sheet of filter paper is deposited on the gel.

During the manufacture of the “sandwich”, care is taken to remove any air bubbles using a glass rod so as not to hamper the transfer. The apparatus is closed with a lid constituting the cathode. The protein transfer is performed at 10V for 90 minutes.

Staining with Ponceau Red

To check the quality of the transfer, the proteins are stained with Ponceau Red (Sigma). The cellulose membrane is rinsed with milliQ water and then soaked once in a bath of Ponceau Red for 10 minutes with stirring. It is then washed in several baths of milliQ water until the coloration remains only on the protein bands. The membrane is placed in a plastic bag and scanned. The protein bands may be quantified to determine the total amount of transferred protein.

Blocking of the Non-Specific Binding Sites

The membrane is stirred overnight at 4° C. or for 90 minutes at room temperature in a solution for blocking the non-specific binding sites, constituted of 5% skimmed milk (Régilait) in PBS-T buffer (cf. Appendix B) (20 ml/membrane).

Immunodetection

The references and the optimum dilutions of the antibodies are given in Appendix C.

After blocking the non-specific sites, the membrane is rapidly rinsed in PBS-T. This membrane is placed in contact with the primary antibody diluted to the optimum concentration in PBS-T with or without 5% milk (m/v) depending on the antibody, for 60 minutes with stirring at room temperature or overnight at 4° C.

It is then rinsed rapidly for three times 10 minutes in PBS-T in order to remove the excess non-bound free antibody. It is then placed in contact with the appropriate secondary antibody coupled to peroxidase, diluted in PBS-T or 5% milk (5 ml) with stirring at room temperature. After incubation for 45 minutes, it is rinsed rapidly twice and then washed 5 times for 5 minutes with PBS-T buffer and a final time in 1×PBS. After draining, it is placed on kitchen cling film (SARAN), protein side upwards.

The membrane is revealed using a highly sensitive chemoluminescence detection kit (Amersham; ECL Western blotting), using luminol as peroxidase substrate. Under the action of peroxidase and an amplifier, the luminol is oxidized and passes into a transient excited state. Return to the ground state takes place by emission of photons, which strike an autoradiography film placed on the membrane.

1 ml of each of the two solutions of the detection kit, i.e. 2 ml, the minimum volume required to cover the membrane, are mixed together. The mixture is immediately poured uniformly onto the membrane and left in contact for exactly one minute at room temperature. The drained membrane is enclosed in Saran cling film and placed in a cassette protected from light, and then covered with a preflashed autoradiography film (Amersham, Hyperfilm ECL). After exposure for 5 minutes, the autoradiography film is revealed. A new film may be exposed if necessary, to optimize the desired signal (up to 1 hour of exposure time). The bands are quantified by means of the Gels Analysts 3.01 software.

5. Mitochondrial aconitase assay by IEF

This technique allows separation of the three forms of mitochondrial aconitase, the active form [4Fe-4S]²⁺, the inactive form [3Fe-4S]⁺ and the apoenzyme form, according to their isoelectric point.

50 μg of mitochondrial protein are deposited onto a pH 3-10 IEF gel (Biorad). Migration is performed in the criterion system (Biorad) for 1 hour at 100V; 1 hour at 250V and 30 minutes at 500V. After migration, the gel is transferred onto a nitrocellulose membrane and anti-mitochondrial aconitase Western blotting is performed in accordance with the protocol described in paragraph 4.

Results

The mitochondrial aconitase activity was measured after isolating the mitochondria from these cell cultures. A decrease in mitochondrial aconitase activity with ageing is demonstrated.

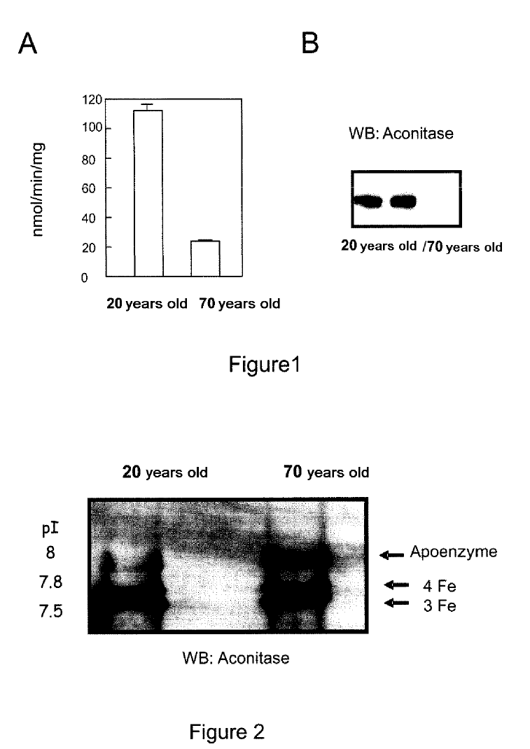

These measurements indicate an approximately 85% decrease in mitochondrial aconitase activity in the mitochondria of the 70-year-old donors in comparison with the 20-year-old donors (cf. FIG. 1).

However, there are no significant differences in expression of the enzyme, i.e. in the amount of enzyme present in the young and old fibroblasts (cf. FIG. 1).

To check whether the decrease in mitochondrial aconitase activity demonstrated in the aged fibroblasts was not due to oxidative damage at the Fe—S centre of the enzyme, the three structural forms of the enzyme (apoenzyme, active form and inactive form) were separated according to their isoelectric point, by isoelectric focusing (IEF). Using this method, a difference with age is demonstrated in the apoenzyme, but there is no difference in the other forms (cf. FIG. 2).

We have shown that the activity of mitochondrial aconitase decreases with age in human dermal fibroblasts in culture. This decrease in activity is not accompanied by a change in expression of the protein with age.

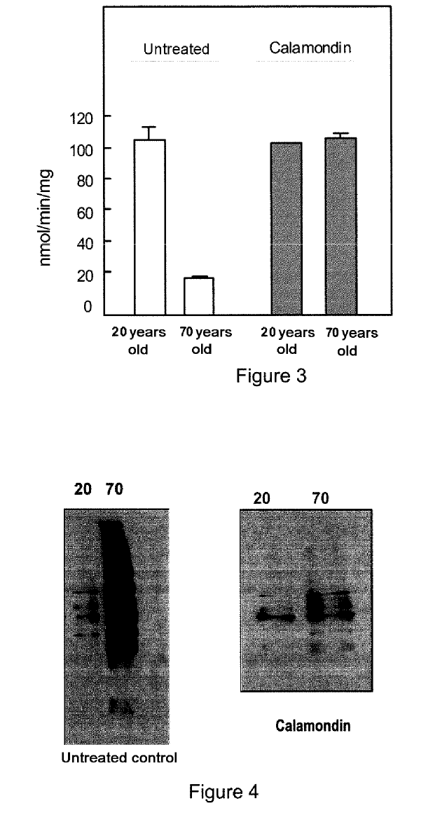

Starting from this result, we characterized the effect of the active agents tested on the mitochondrial aconitase activity in dermal fibroblasts obtained from these donors of different age (cf. FIG. 3 and Table 1).

The results are expressed as a percentage relative to the mitochondrial aconitase activity in untreated fibroblasts from a young donor (20 years old), which constitutes the 100% level.

TABLE 1
Mitochondrial aconitase activity of fibroblasts from 20-
and 70-year-old donors

| (%) | Untreated | Calamondin |
|---|---|---|
| 20 years old | 100 | 97 |
| 70 years old | 18 | 108 |

These results indicate that a treatment with these extracts makes it possible to protect the mitochondrial aconitase against the action of oxidizing agents. However, no significant activation of mitochondrial aconitase is observed, 48 hours after treating the fibroblasts from 20-year-old donors with these extracts. On the other hand, the same treatment on fibroblasts from 70-year-old donors results in total protection of the enzyme.

### Example 3

### Revelation of Oxidized Proteins by Oxy Blotting

a—Principle

The carbonyl groups (oxidation markers) introduced into the protein chains by means of ROS or other oxidation mechanisms such as glycation/glycoxidation or lipoperoxidation, is revealed according to a site-specific mechanism.

The carbonyl groups in the chains react with 2,4-dinitrophenylhydrazine (DNPH) to give a hydrazone derivative. The DNP-derived samples are separated out on a polyacrylamide gel by electrophoresis. The separation is followed by transfer onto a nitrocellulose membrane as for Western blotting. The membrane is then incubated in the presence of the first antibody, specific for the molecule DNP bound to the proteins bearing a carbonyl group. The next step is incubation with the anti-primary antibody (anti-rabbit) secondary antibody which is coupled to peroxidase. The revelation is performed using the same reagents used in Western blotting.

b—Protocol

All the reagents used are from the Oxyblot kit (Appligene-Oncor, France).

Denaturing of the Samples

An amount of protein of between 15-20 μg is used, corresponding to 2 μg of deposit originating from the mitochondrial lysates contained in 5 μl of cell extract obtained according to Example 2 (Materials and Methods, paragraph 1). 10 μl of 1×DNPH are added, followed by 5 μl of 12% SDS; the mixture is stirred for 15 minutes at room temperature. 7.5 μl of neutralizing solution and the 1× sample buffer are added. The samples are ready to be deposited.

The proteins are finally separated by electrophoresis on 12% SDS acrylamide and transferred onto nitrocellulose membrane.

Antigen-Antibody Reaction

The carbonyl derivatives are revealed with an anti-DNP rabbit primary antibody diluted 150-fold and an anti-rabbit secondary antibody. The ECL kit (Amersham) is used to reveal the peroxidase with the aid of its substrate.

Results

An increase in oxidized proteins with age is observed (FIG. 4). However, treatment of the aged fibroblasts with the calamondin extract makes it possible to return to a level of oxidized proteins corresponding to that of young fibroblasts. These results demonstrate that the abovementioned extract has antioxidant activity on oxidized proteins of the skin.

### Examples of Cosmetic Formulations

### Example 4

### Cosmetic Composition Comprising an Extract of Calamondin Fruit

The extract obtained in Example 1 is used in the form as obtained, in the cosmetic composition below:

| | |
|---|---|
| Plant extract of Calamondin or Calamansis (Ex. 1) | 0.1% |
| Surfactant (Arlacel ® 165 VP) | 5% |
| 95% cetyl alcohol | 1% |
| Stearyl alcohol | 1% |
| Beeswax | 1.5% |
| Oil (Parleam ®) | 8.5% |
| Glyceride tricaprate/caprylate | 3% |
| Silicone oil (Dimethicone 100 CS) | 1% |
| Polymer (Keltrol ®) | 0.35% |
| Sodium hydroxide | 0.04% |
| Tetrasodium EDTA powder | 0.1% |
| Preserving agents | 0.5% |
| Water | qs 100 |

The cosmetic composition is prepared in the usual manner, well known to those skilled in the art, by mixing together the various components in one or more steps.

This composition may be applied to facial skin daily for several weeks to obtain the anti-ageing cosmetic effects indicated previously, and to restore a radiant complexion.

### Appendix A

### I—Buffers and Solutions Used for the Electrophoresis Gels Under Denaturing and Reductive Conditions, in Discontinuous Buffer

Monomer Solution:

acrylamide/bisacrylamide, 30% T, 2.67% C (Biorad)

Resolution gel buffer: Tris-HCl 1.5M pH 8.8

18.15 g of Tris base (Sigma) per 100 ml of distilled water
pH adjusted to 8.8 with 12N HCl

Concentrating gel buffer: 0.5M Tris-HCl pH 6.8

6 g of Tris base per 100 ml of distilled water
pH adjusted to 6.8 with 12N HCl

10× migration buffer: 0.25M Tris pH 8.3, 1.92M glycine; 1% SDS

| | | |
|---|---|---|
| Tris base | 12 | g |
| Glycine (Research Organics Inc) | 57.6 | g |
| 10% SDS (Sigma) | 40 | ml |
| Distilled water | qs 400 | ml |

Ammonium persulfate: (NH₄)₂S₂O₈: (Sigma) at 10% (w/v), i.e. 100 mg/ml

2× Laemmli reductive sample buffer: 0.06M Tris-HCl pH 6.8; 2.3% SDS; 10% glycerol: 0.02% bromophenol blue

| | | |
|---|---|---|
| pH 6.8 0.5M Tris concentrating gel buffer | 6.25 | ml |
| 10% SDS | 11.50 | ml |
| Glycerol | 5 | ml |
| Distilled water | qs 50 | ml |

10× Bromophenol Blue (Saturated Solution):

A spatula-tip of bromophenol blue is dispersed in 5 ml of 2× Laemmli buffer. After stirring, sonicating and then centrifuging, the supernatant is recovered.

### II—Electrophoresis Gels

Preparation of the 12% T resolution gel

| | Volumes | | Final |
|---|---|---|---|
| Solutions | for 2 gels (10 ml) | | concentrations |
| Monomer solution | 4.0 | ml | 12% T; 2.7% C |
| Resolution gel buffer | 2.5 | ml | 0.375M |
| 10% SDS | 100 | μl | 0.1% |
| Ammonium persulfate (10%) | 50 | μl | 0.05% |
| TEMED (Research Organics Inc.) | 5 | μl | — |
| Distilled water | 3.4 | ml | — |

Preparation of the R % T concentrating gel

| | Volumes | | Final |
|---|---|---|---|
| Solutions | for 2 gels (5 ml) | | concentrations |
| Monomer solution | 2 | ml | 8% T; 2.7% C |
| Resolution gel buffer | 1.25 | ml | 0.375M |
| 10% SDS | 50 | μl | 0.1% |
| Ammonium persulfate (10%) | 25 | μl | 0.05% |
| TEMED (Research Organics Inc.) | 5 | μl | — |
| Distilled water | 4.2 | ml | — |

### Appendix B

### Solutions for the Transfer and Immunodetection

Towbin Transfer Buffer:

25 mM Tris-HCl, pH 8.3; 192 mM glycine; 20% methanol

| | | |
|---|---|---|
| Tris base | 3.03 | g |
| Glycine (Research Organics Inc.) | 14.4 | g |
| to be dissolved in 100 ml of distilled water | | |
| Methanol | 200 | ml |
| Distilled water | qs 1000 | ml |

PBS-T Buffer

Tenfold dilution of 10×PBS (Invitrogen)

+0.1% Tween 20 (Sigma)

Ponceau Red (Sigma)

0.1% (w/v) solution in 5% acetic acid solution

### Appendix C

### List of Primary and Secondary Antibodies

| | | | Incubation |
|---|---|---|---|
| Antibody | Reference | Dilution | time |
| Human anti-aconitase | not commercial | 1/1000 | 1 hour |
| | (cf. ref. below) | | |
| Anti-rabbit IgG HRP | Amersham | 1/50 000 | 1 hour |

Ref.: Bulteau et al. Biochemistry, 2003; 42, 14846-14855

1. A method of cosmetic care for preventing or retarding the appearance of the signs of ageing on the dermis or for attenuating the effects thereof, said method comprising the delivery to at least one area of bodily or facial dermis of an effective amount of at least one cosmetically acceptable active agent, said active agent being a plant extract obtained from a plant belonging to the genus Citrus, or a hybrid derived from the crossing of plant species, at least one of which belongs to the genus Citrus.

2. The method according to claim 1, wherein the extract is obtained from a plant belonging to the genus×Citrofortunella.

3. The method according to claim 1, wherein the extract is obtained from calamondin (×Citrofortunella microcarpa).

4. The method according to claim 1, wherein said extract is obtained from a part of said plant, said plant part being selected from the group consisting of the root, the rhizome, an aerial part, the stem, the leaves, the flowers, the seeds, the fruit, the floral buds, the whole fruit, and a part of the fruit.

5. The method according to claim 1, wherein the extract is obtained from calamondin fruit.

6. The method according to claim 5, wherein said extract is obtained from a polar solvent or a mixture of polar solvents extraction from the fruit juice of said calamondin fruit.

7. The method according to claim 6, wherein said polar solvent or the mixture of polar solvents used for the extraction is selected from the group consisting of water, a C₁-C₄ alcohol, ethanol, butanol, a glycol, glycerol, butylene glycol, propylene glycol, polyglycerol-3, and mixtures thereof.

8. The method according to claim 7, wherein said mixtures are mixtures of at least one alcohol and water or of at least one glycol and water, comprising at least 10 v/v % of alcohol or glycol, the remainder being water.

9. The method according to claim 8, wherein said solvent mixture comprises a 50/50 v/v ratio water and ethanol mixture or a 50/50 v/v ratio water and butylene glycol mixture.

10. The method according to claim 7, wherein said extraction is performed in aqueous or water-glycol medium.

11. The method according to claim 1, wherein said extract is in the form of a powder.

12. The method according to claim 1, wherein said active agent, incorporated into a cosmetic composition also comprising at least one cosmetically acceptable excipient, and wherein said active agent is delivered topically by applying said composition to at least one concerned area of bodily or facial skin.

13. The method according to claim 1, wherein the concentration of said active agent is between 0.001% and 5% by weight of the cosmetic composition.

14. A cosmetic composition comprising at least one cosmetically acceptable active agent and at least one cosmetically acceptable excipient, wherein said active agent is a plant extract obtained from at least one plant belonging to the genus×Citrofortunella.

15. The composition according to claim 14, wherein the plant extract is an extract of calamondin (×Citrofortunella microcarpa) optionally used in combination with at least one other active agent that participates in the maintenance and/or integrity of the structure of the skin.

16. The composition according to claim 14, wherein said extract is an extract from a part of said plant, said plant part being selected from the group consisting of the root, the rhizome, an aerial part, the stem, the leaves, the flowers, the seeds, the fruit, the floral buds, the whole fruit, and a part of the fruit.

17. The composition according to claim 14, wherein said extract is a dried or ground plant material extract; or a freshly harvested plant material extract.

18. The composition according to claim 14, wherein said extract is a calamondin fruit extract.

19. The composition according to claim 14, wherein said extract is a polar solvent or a mixture of polar solvents extract from the fruit juice of said calamondin fruit.

20. The composition according to claim 19, wherein said polar solvent or the mixture of polar solvents used for the extraction is selected from the group consisting of water, a C₁-C₄ alcohol, ethanol, butanol, a glycol, glycerol, butylene glycol, propylene glycol, polyglycerol-3, and mixtures thereof.

21. The composition according to claim 20, wherein said mixture is a mixture of at least one alcohol and water or of at least one glycol and water, comprising at least 10 v/v % of alcohol or glycol, the remainder being water.

22. The composition according to claim 21, wherein the solvent mixture comprises a 50/50 v/v ratio water and ethanol mixture or a 50/50 v/v ratio water and butylene glycol mixture.

23. The composition according to claim 14, wherein said extract is at a concentration of between 0.001% and 5% by dry weight of extract relative to the composition.

24. The composition according to claim 14, wherein said composition comprises at least one cosmetically acceptable excipient chosen from pigments, dyes, polymers, surfactants, rheological agents, fragrances, electrolytes, pH modifiers, antioxidants and preserving agents, and mixtures thereof.

25. The composition according to claim 14, wherein said composition is formulated in the form of a serum, a lotion, an emulsion, a cream, a hydrogel, a mask, a stick, or a patch.
